(19) United States
(12) Patent Application Publication
(10) Pub. No.: US 2008/0241272 A1
(43) Pub. Date: Oct. 2, 2008

(54) Composition for Prevention and Treatment of Stretch Mark Comprising Citric Acid, Zinc and Arginine

(76) Inventors: Seog-Nyeon Bae, Seoul (KR); Lae-Ok Park, Seoul (KR)

Correspondence Address: SHERIDAN ROSS PC, 1560 BROADWAY, SUITE 1200, DENVER, CO, 80202, US

(21) Appl. No.: 11/816,901

(22) PCT Filed: Feb. 25, 2005
(86) PCT No.: PCT/KR05/00527

§ 371 Date: Dec. 21, 2007

(30) Foreign Application Priority Data
Feb. 23, 2005 (KR) 10-2005-0014848

(51) Int. Cl. A61K 33/30; A61P 17/00

(52) U.S. Cl. 424/642

(57) ABSTRACT

Disclosed is a composition for preventing and treating stretch marks, including citric acid, zinc and arginine as active components. The composition is applicable to lesions after being formulated as a topically applied medicament or cosmetic product. The active components of the present invention, which are easily obtainable from natural sources, are safe to the body and are capable of preventing and treating stretch marks even in small amounts.

### TECHNICAL FIELD

The present invention relates to a composition for preventing and treating stretch marks, and more particularly, to a composition for preventing and treating stretch marks comprising citric acid, zinc and arginine as active components. The composition is applicable to lesions after being formulated as a topically applied medicament or cosmetic product

### BACKGROUND ART

Stretch marks, also known medically as stria atrophica and striae distensae, result from tears in the skin of the thigh, abdomen, etc., which occur when the skin becomes thinner. Generally, stretch marks develop because the skin tissue is disrupted when the skin is excessively stretched during sudden or excessive weight gain. However, stretch marks can even occur without sudden or excessive weight gain. Stretch marks can occur due to excess production of growth hormone, pregnancy, exercise such as weight lifting, or disorders of the endocrine system. Also, stretch marks can develop from the long-term use of topical steroid drugs. Stretch marks are located at various areas of the body according to causes. They occur mainly in the legs during puberty or rapid weight gain, mainly in the lower abdomen and thighs during pregnancy, and anywhere on the body from endocrine disorders.

Stretch marks start as red lines or bands in the thighs, buttocks, lower abdomen, the area on the back side of the leg at the knee joint, and breast, in which the outer layer of the skin is shrunken and elastic fibers are lost in the deep skin layer (dermis). The lines are slightly depressed relative to the surrounding normal skin, and thus, unevenness is detected when touched by hands. As time goes by, the color of the early lines changes to white.

It is important to initiate treatment as early as possible. Treatment is relatively effective in early stages, but satisfactory treatment results are difficult to achieve in later stages. Typical treatment in the early stages of stretch marks is to induce the color change using a versa pulse laser, and then apply retinoic acid topically. In the later stages, stretch marks are treated with topical application of retinoic acid for several months in combination with CO₂ laser or Erbium YAG laser treatment about three times at intervals of three to four months. However, retinoic acid has side effects including skin dryness and birth defects, and laser treatment is expensive and may cause pigmentation in some cases.

On the other hand, Korean Patent Laid-open Publication No. 2001-0097020 discloses a composition for treating stretch marks comprising an extract of Centella asiatica, and more particularly, a novel externally applied niosome-multiple emulsion, which is prepared by adding a surfactant and a lipid to a C. asiatica extract containing about 40% asiaticicoside, about 30% madecassic acid and about 30% asiatic acid to generate niosomes, mixing ceramide with a vegetable oil, labrafac, etc. to fonn an oily solution, and mixing the oily solution with the niosomes.

In addition, International Patent Publication No. WO 2003/004043 discloses a composition for treating various types of scars including stretch marks comprising an onion extract. The onion extract is obtained by first extracting dried onions with an extractant by percolating the dried onions at 40-90° C., and evaporating the percolate at above 30° C. under pressure.

However, the above compositions are disadvantageous because they are prepared by a complicated process, which includes an extraction step to obtain active components.

### DISCLOSURE OF THE INVENTION

The present inventors found that a composition comprising citric acid, zinc and arginine, which are commercially available without the extraction step, is capable of preventing and treating stretch marks by stimulating the growth of keratinocytes and fibroblasts and increasing collagen synthesis.

In one aspect, the present invention provides a topically applied pharmaceutical composition for preventing and treating stretch marks, which is characterized by comprising citric acid, zinc and arginine as active components along with a pharmaceutically acceptable carrier.

In another aspect, the present invention provides a cosmetic composition, which is characterized by comprising citric acid, zinc and arginine as active components along with a cosmetically acceptable carrier.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing the effect of a composition, for preventing and treating stretch marks according to the present invention, on keratinocyte proliferation in various concentrations;

FIG. 2 is a graph showing the effect of a composition, for preventing and treating stretch marks according to the present invention, on collagen synthesis in various concentrations;

FIG. 3 is a graph showing the effect of a composition, for preventing and treating stretch marks according to the present invention, on fibroblast proliferation in various concentrations;

FIG. 4a shows the state of the skin where stretch marks occur before a cosmetic composition according to the present invention is applied to the skin;

FIG. 4b shows the state of the skin where stretch marks occur after a cosmetic composition according to the present invention is applied to the skin;

FIG. 5a shows the state of the skin where stretch marks occur before a cosmetic composition according to the present invention is applied to the skin;

FIG. 5b shows the state of the skin where stretch marks occur after a cosmetic composition according to the present invention is applied to the skin;

FIG. 6a shows the state of the skin where stretch marks occur before a cosmetic composition according to the present invention is applied to the skin;

FIG. 6b shows the state of the skin where stretch marks occur after a cosmetic composition according to the present invention is applied to the skin;

FIG. 7a shows the state of the skin where stretch marks occur before a cosmetic composition according to the present invention is applied to the skin;

FIG. 7b shows the state of the skin where stretch marks occur after a cosmetic composition according to the present invention is applied to the skin;

FIG. 8a shows the state of the skin where stretch marks occur before a cosmetic composition according to the present invention is applied to the skin;

FIG. 8b shows the state of the skin where stretch marks occur after a cosmetic composition according to the present invention is applied to the skin;

FIG. 9a shows the state of the skin where stretch marks occur before a cosmetic composition according to the present invention is applied to the skin;

FIG. 9b shows the state of the skin where stretch marks occur while a cosmetic composition according to the present invention is applied to the skin;

FIG. 9c shows the state of the skin where stretch marks occur after a cosmetic composition according to the present invention is applied to the skin;

FIG. 10a shows the state of the skin where stretch marks occur before a cosmetic composition according to the present invention is applied to the skin;

FIG. 10b shows the state of the skin where stretch marks occur while a cosmetic composition according to the present invention is applied to the skin; and

FIG. 10c shows the state of the skin where stretch marks occur after a cosmetic composition according to the present invention is applied to the skin.

### BEST MODE FOR CARRYING OUT THE INVENTION

The present invention relates to a composition for preventing and treating stretch marks, comprising citric acid, zinc and arginine as active components. Since the composition has preventive and therapeutic efficacy on stretch marks that occur on the skin, it may be used in a medical or cosmetic formulation, which is topically applied onto the skin. Hereinafter, the present invention will be described in detail.

In one aspect, the present invention provides a topically applied pharmaceutical composition for preventing and treating stretch marks, comprising citric acid, zinc and arginine as active components along with a pharmaceutically acceptable carrier.

The pharmaceutical composition comprises citric acid in an amount of 0.01 to 20% by weight, preferably 0.5 to 5% by weight, based on the total weight of the composition, zinc in an amount of 0.001 to 5% by weight, preferably 0.01 to 1% by weight, based on the total weight of the composition, and arginine in an amount of 0.01 to 20% by weight, preferably 0.5 to 10% by weight, based on the total weight of the composition, along with a pharmaceutically acceptable carrier in the remaining amount to total 100% by weight.

Citric acid, zinc and arginine, used as active components of the pharmaceutical composition, may be contained in the topically applied pharmaceutical composition according to the present invention in the forms described below, but the present invention is not limited to the following examples.

Citric acid may be contained in a free acid or salt form, which is generally commercially available. Citric acid may be in any of the forms extracted from seeds or fruit juice of a variety of plants, in which citric acid exists in a free form. Also, citric acid prepared by a surface fermentation process or submerged fermentation process using the fungus Aspergillus niger may be used in the composition of the present invention. In the process of preparing citric acid, fermentation is performed using molasses as a source of fermentable sugar, and various centrifugal separators, including a separator with nozzle bowl, a separator with a self-cleaning bowl and a decanter, are used for isolating citric acid.

Arginine may be contained in a free base or salt form, which is generally commercially available. Preferably arginine may be in any of the forms prepared by fermentation or extraction from protein sources. Examples of methods of preparing arginine by fermentation include fermentation using a microorganism resistant to arginine analogues (Agricultural Biological Chemistry, 1972, Vol. 36, p 339; Journal of General Applied Microbiology, 1973, Vol. 19, p 339; Japanese Patent Publication No. 3391/1973 and U.S. Pat. No. 3,723,249), production of arginine directly from carbon and nitrogen sources, for example, by use of a glutaminic acid-producing microorganism belonging to the genus Brevibacterium or Corynebacterium (Japanese Patent Laid-open Publication Nos. Sho57-1634875, Sho60-83593 and Sho62-2659885), use of an amino acid-producing microorganism having an improved growth property through cell fusion (Japanese Patent Laid-open Publication No. Sho58-158185), and use of a microorganism transformed with a recombinant expression vector carrying a gene encoding an enzyme participating in the biosynthesis pathway of arginine (Japanese Patent Laid-open Publication No. Sho63-79597, Japanese Patent Publication No. 66989/1985 and U.S. Pat. No. 4,775,623).

Zinc useful in the present invention may be in any of the forms extracted from animal products including oysters, crusts, fishes and red meat, and various vegetable products including grain, beans, nuts and seeds, in which zinc is present in abundance. Non-limiting examples of forms of zinc applicable to the present composition include chelated zinc ions with picolinate, acetate, sodium citrate, glycerate and monomethionine. Generally, these forms of zinc are also commercially available.

On the other hand, the pharmaceutical composition according to the present invention may be formulated into pharmaceutical preparations typical for topical application, including ointments, gels, creams, liniments and lotions. The type and concentration of a pharmaceutically acceptable carrier may vary according to desired formulations, but may be readily determined by those skilled in the art.

In an aspect of formulation, the pharmaceutical composition may be formulated into an ointment, which may be prepared by combining and processing ointment base materials, active components and other additives typically contained in other ointments at a suitable ratio. The ointment base materials are selected from among those known in the art, which are exemplified by higher fatty acids or esters thereof (e.g., adipic acid, myristic acid, palmitic acid, stearic acid, oleic acid, adipic acid esters, myristic acid esters, palmitatic acid esters, dimethyl sebacate, hexyl laurate, cetyl octanate, etc.), waxes (e.g., spermaceti, beeswax, cerecine, etc.), surfactants (e.g., polyoxyethylene alkylether phosphate esters, etc.), higher alcohols (e.g., cetanol, stearylalcohol, cetostearylalcohol, etc.), silicon oils (e.g., dimethyl polysiloxanes, methylphenyl polysiloxanes, glycol methyl polysiloxanes, silicon glycol copolymers, etc.), hydrocarbons (e.g., hydrophilic petrolatum, white petrolatum, purified lanolin, liquid paraffin, etc.), water, humectants (e.g., glycerin, propylene glycol, butylene glycol, sorbitol, etc.), and infection inhibitors.

In another aspect of formulation, the pharmaceutical composition may be formulated into a gel, which may be prepared by combining and processing gel base materials, active components and other additives typically contained in other gels at a suitable ratio. The gel base materials are selected from among those known in the art which are exemplified by gelling agents (e.g., carboxyvinyl polymers, hydroxyethyl cellulose, ethyl cellulose, carboxymethyl cellulose, propylene glycol alginate esters, etc.), alcohols (e.g., ethanol, isopropyl alcohol, etc.), water, neutralizing agents (e.g., triethanolamine, diisopropanolamine, sodium hydroxide, etc.), surfactants (e.g., sorbitane sesquioleate, sorbitane trioleate, sorbitane monooleate, sorbitane monostearate, sorbitane monolaurate, polyethylene glycol monostearate, polyoxyethylene nonyl phenylether, polyoxyethylene laurylether, etc.), and infection inhibitors.

In a further aspect of formulation, the pharmaceutical composition may be formulated into a cream, which may be prepared by combining and processing cream base materials, active components and other additives typically contained in other creams at a suitable ratio. The cream base materials are selected from among those known in the art which are exemplified by higher fatty acid esters (e.g., myristic acid esters, palmitic acid esters, dimethyl sebacate, hexyl laurate, cetyl isooctanate, etc.), lower alcohols (e.g., ethanol, isopropanol, etc.), carbohydrates (e.g., liquid paraffin, squalane, etc.), multivalent alcohols (e.g., propylene glycol, 1,3-butylene glycol, etc.), higher alcohols (e.g., 2-hexyldecanol, cetanol, 2-octyldecanol, etc.), emulsifying agents (e.g., polyoxyethylene alkylethers, fatty acid esters, polyethylene glycol fatty acid esters, etc.), antiseptics (e.g., paraoxybenzoic acid esters, etc.), and infection inhibitors.

In still another aspect of formulation, the pharmaceutical composition may be formulated into a liniment, which may be prepared by suitably combining and processing an active component with alcohols (e.g., monovalent alcohols such as ethanol, propanol and isopropanol, multivalent alcohols such as polyethylene glycol, propylene glycol and butylene glycol, etc.), water, fatty acid esters (e.g., various esters of adipic acid, sebacic acid, myristic acid, etc.), surfactants (e.g., polyoxyethylene alkylethers, etc.), if desired, in combination with ultraviolet absorbers, antioxidants, neutralizers for controlling pH, viscosity controlling agents (e.g., methyl cellulose, carboxyvinyl polymers, hydroxypropyl cellulose, etc.), and infection inhibitors.

In still another aspect of formulation, the pharmaceutical composition may be formulated into an aerosol, which may be prepared using a liquid or suspension of an active component and a propellant. The liquid or suspension may be prepared using a diluting agent typically used in the art or used in the preparation of injectable preparations. Typically used propellants may be used, which are exemplified by liquefied gas propellants including chlorofluorocarbons, such as freon-12 (chlorodifluoromethane) and freon-123 (trifluorodichloroethane), and compressed gas propellants including nitrogen and carbon dioxide gases. An aerosol may include a solubilizer and a buffering agent, which are typically used in the art, and, if desired, may further include a colorant, a preservative, an aromatic and a flavoring agent.

The effective daily dosage of the pharmaceutical composition according to the present invention when topically applied ranges from about 0.1 μg to about 500 mg per kg body weight, preferably about 1 μg to about 250 mg per kg body weight, and most preferably about 2 μg to about 100 mg per kg body weight. It is apparent to those skilled in the art that the specific amount of the pharmaceutical composition to be topically applied to a patient may vary depending on a variety of factors including the patient's symptoms and resistance to drugs.

In another aspect, the present invention provides a cosmetic composition comprising citric acid, zinc and arginine as active components along with a cosmetically acceptable carrier.

The cosmetic composition comprises citric acid in an amount of 0.01 to 20% by weight, preferably 0.5 to 5% by weight, based on the total weight of the composition, zinc in an amount of 0.001 to 5% by weight, preferably 0.01 to 1% by weight, based on the total weight of the composition, and arginine in an amount of 0.01 to 20% by weight, preferably 0.5 to 10% by weight, based on the total weight of the composition, along with a cosmetically acceptable carrier in the remaining amount to total 100% by weight.

Arginine, citric acid and zinc, used as active components in the cosmetic composition, may be contained in the same forms as in the aforementioned pharmaceutical composition.

On the other hand, the “cosmetically acceptable carrier” includes, but is not limited to, purified water, oils, waxes, fatty acids, fatty acid alcohols, fatty acid esters, surfactants, humectants, thickening agents, antioxidants, viscosity stabilizers, chelating agents, buffering agents, antiseptics, and lower alcohols. If desired, the carrier may include whitening agents, moisturizing agents, anti-inflammatory agents, antibacterial agents, antifungal agents, vitamins, UV screening agents, antibiotics, anti-pimple agents, perfumes, and dyes.

In detail, available oils may include hydrogenated vegetable oils, castor oil, cottonseed oil, olive oil, palm oil, jojoba oil and avocado oil. Available waxes may include beeswax, spermaceti, camauba, candelilla, montan, cerecine, liquid paraffin and lanolin. Available fatty acids may include stearic acid, linoleic acid, linolenic acid and oleic acid, and available fatty acid alcohols may include cetyl alcohol, octyl dodecanol, oleyl alcohol, panthenol, lanolin alcohol, stearyl alcohol and hexadecanol. Available fatty acid esters may include isopropyl myristate, isopropyl palmitate and butyl stearate. However, the present invention is not limited to these examples.

Non-limiting examples of the surfactants include anionic surfactants, such as sodium stearate, sodium cetylsulfate, polyoxyethylene laurylether phosphate and sodium N-acyl glutamate; cationic surfactants, such as stearyldimethyl-benzylammonium chloride and stearyltrimethylammonium chloride; amphoteric surfactants, such as alkylaminoethylglycine hydrochloride and lecithin; and nonionic surfactants, such as glycerin monostearate, sorbitan monostearate, sucrose fatty acid ester, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol, polyoxyethylene castor oil and polyoxyethylene lanolin.

Non-limiting examples of the humectants include glycerin, 1,3-butylene glycol and propylene glycol. Available lower alcohols include ethanol and isopropanol. Non-limiting examples of the thickening agents include sodium alginate, sodium caseinate, gelatin agar, xanthan gum, starch, cellulose ethers (e.g., hydroxyethyl cellulose, methyl cellulose, carboxymethyl cellulose, hydroxy propylmethyl cellulose), polyvinylpyrrolidone, polyvinylalcohol, polyethylene glycol and sodium carboxymethyl cellulose. Non-limiting examples of the antioxidants include butylated hydroxytoluene, butylated hydroxyanisole, propyl gallate, citric acid and ethoxyquin. Non-limiting examples of the chelating agents include disodium edetate and ethane-hydroxy diphosphate. Non-limiting examples of the buffering agents include citric acid, sodium citrate, boric acid, borax and disodium hydrogen phosphate. Non-limiting examples of the antiseptics include methyl parahydroxybenzoate, ethyl parahydroxybenzoate, dehydroacetic acid, salicyic acid and benzoic acid.

The cosmetic composition according to the present invention may be useful especially for pregnant women who are prone to develop stretch marks but must not use drugs thoughtlessly, or patients having stretch marks over a wide range of areas of the skin.

A better understanding of the present invention may be obtained through the following examples which are set forth to illustrate, but are not to be construed as the limit of the present invention.

### EXAMPLE 1

### Preparation of a Composition for Preventing and Treating Stretch Marks According to the Present Invention

2 wt % of citric acid (Sigma, USA), 0.1 wt % of zinc (Sigma, USA), 5 wt % of arginine (Sigma, USA) and hydroxyethylcellulose in the remaining amount to total 100% were mixed thoroughly in a suitable container. Then, a composition for preventing and treating stretch marks according to the present invention was prepared from formed precipitates.

### EXAMPLE 2

### Evaluation of the Effect of the Composition of the Present Invention on Proliferation of Keratinocytes

The present composition for preventing and treating stretch marks was evaluated for its effect on keratinocyte proliferation by measuring cell viability of keratinocytes. High cell viability indicates that keratinocyte proliferation is stimulated.

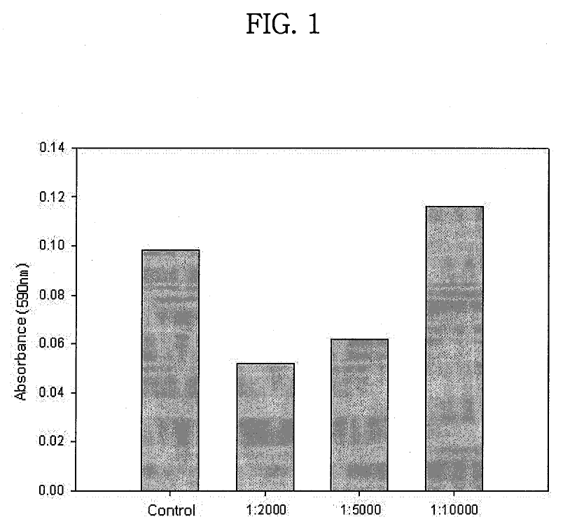

1×10³ cells were plated on each well of 24-well plates, and over 80% of them were attached onto the bottom of the plates. The composition for preventing and treating stretch marks, prepared in Example 1, was diluted to 1:2000, 1:5000 and 1:10000, and each dilution was added to each well once every two days for a culture period of 8 days. Subsequently, an MT solution was added to each well, and the cells were further cultured for 4 hrs. Then, a lysis buffer was added to each well, and the plates were allowed to react for 24 hrs. Absorbance was measured at 590 nm, and the results are given in Table 1, below, and FIG. 1.

TABLE 1
Viability of keratinocytes according to concentrations of the
present composition

| | | Composition for preventing | | |
|---|---|---|---|---|
| | Control | and treating stretch marks | | |
| | FAD | 1:2000 | 1:5000 | 1:10000 |
| 1 | 0.099 | 0.052 | 0.059 | 0.109 |
| 2 | 0.092 | 0.051 | 0.064 | 0.110 |
| 3 | 0.103 | 0.054 | 0.064 | 0.128 |
| Mean | 0.098 | 0.052 | 0.062 | 0.116 |

Compared to the control, when keratinocytes were treated with a 1:10000 dilution of the present composition for preventing and treating stretch marks, their viability was increased by about 20%. These results indicate that the present composition for preventing and treating etch marks stimulates the proliferation of keratinocytes at low concentrations.

### EXAMPLE 3

### Evaluation of the Effect of the Composition of the Present Invention on Collagen Synthesis

The present composition for preventing and treating stretch marks was examined for its effect on collagen synthesis.

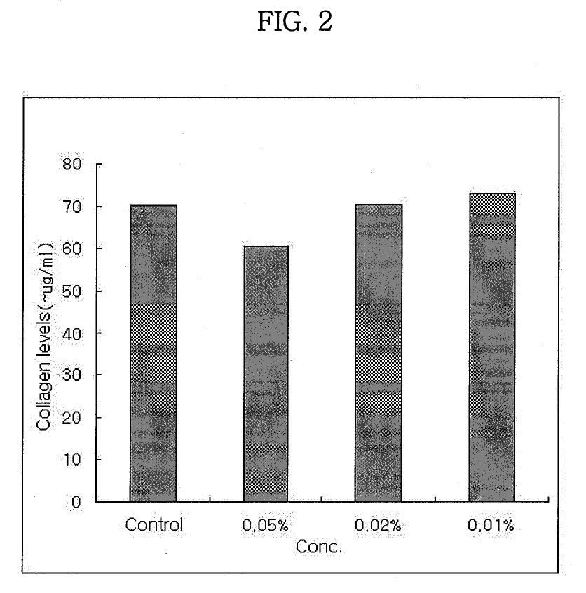

After fibroblasts were cultured for 24 hrs in 24-well plates, the medium was exchanged with a medium supplemented with the composition for preventing and treating stretch marks, prepared in Example 1, and the cells were further cultured for 24 hrs. Then, the culture supematants were mixed with a sircol dye and centrifuged for 10 min. After the supematant was discarded, the pellet was mixed with an alkali reagent and resuspended therein by sufficient vortexing. Absorbance was measured at 540 nm, and collagen concentrations in the culture supematants were determined using a standard curve. The results are given in Table 2, below, and FIG. 2.

TABLE 2
Collagen levels according to concentrations of the
present composition

| | |
|---|---|
| Collagen | |
| levels | |
| (μg/ml) | Mean ± SD |

| Concentrations | 1 | 2 | (μg/ml) |
|---|---|---|---|
| 0% (Control) | 70.9 | 69.5 | 70.2 ± 1.02 |
| 0.05% | 61.2 | 60.0 | 60.6 ± 0.83 |
| 0.02% | 67.6 | 73.0 | 70.3 ± 3.86 |
| 0.01% | 72.0 | 74.1 | 73.1 ± 1.48 |

Compared to the control, the present composition for preventing and treating stretch marks was found to increase collagen synthesis by about 0.01% to 4%.

### EXAMPLE 4

### Evaluation of the Effect of the Composition of the Present Invention on the Proliferation of Fibroblasts

The present composition for preventing and treating stretch marks was examined for its effect on fibroblast proliferation by measuring the viability of fibroblasts. High cell viability indicates that fibroblasts proliferation is stimulated.

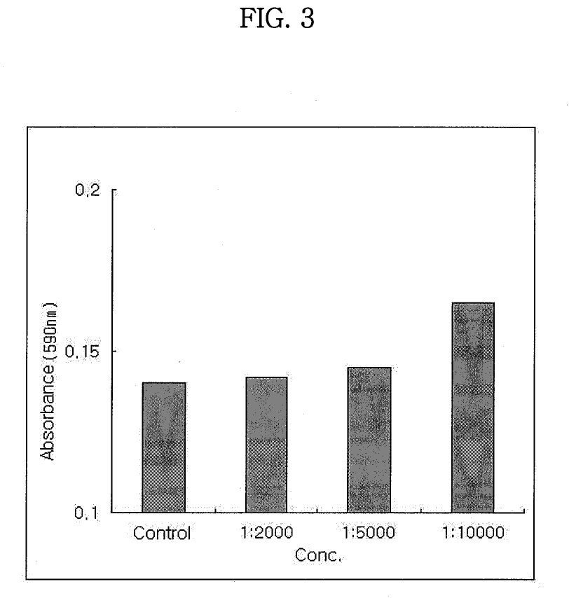

After fibroblasts were cultured for 24 hrs in 24-well plates, the medium was exchanged with a medium supplemented with the composition prepared in Example 1, and the cells were further cultured for 24 hrs. Subsequently, 50 μl of an MTT solution was added to each well, and the cells were further cultured for 4 hrs. Then, 1 ml of a cell lysis buffer was added to each well, and the plates were allowed to react overnight. Absorbance was measured at 590 nm, and cell number was calculated according to a standard curve. The results are given in Table 3, below, and FIG. 3.

TABLE 3
Viability of fibroblasts according to concentrations of the
present composition

| | | Composition for preventing | | |
|---|---|---|---|---|
| | Control | and treating stretch marks | | |
| | DMEM | 1:2000 | 1:5000 | 1:10000 |
| 1 | 0.139 | 0.145 | 0.142 | 0.178 |
| 2 | 0.140 | 0.148 | 0.158 | 0.115 |
| 3 | 0.141 | 0.134 | 0.135 | 0.167 |
| Mean | 0.140 | 0.142 | 0.145 | 0.165 |

Compared to the control, when fibroblasts were treated with a 1:10000 dilution of the present composition for preventing and treating stretch marks, their viability was increased by about 20%. These results indicate that the present composition for preventing and treating stretch marks stimulates the proliferation of fibroblasts at low concentrations.

### EXAMPLE 5

### Preparation of a Cosmetic Composition According to the Present Invention

5 g of citric acid, 0.8 g of zinc, 5 g of arginine, 75 g of distilled water, 5 g of glycerin as a humectant, 0.05 g of EDTA as a chelating agent, and 0.15 g of KOH as a thickening agent were mixed thoroughly in a beaker at about 70° C., thus generating an aqueous phase. Separately, 4 g of olive oil, 3 g of stearic acid and 3 g of a surfactant were mixed thoroughly in a beaker at about 70° C., thus generating an oil phase. The aqueous phase was mixed with the oil phase and sufficiently dispersed by agitation at 7000-8000 rpm, thereby yielding a cosmetic composition according to the present invention.

### EXAMPLE 6

### Evaluation of the Therapeutic Effect of the Cosmetic Composition According to the Present Invention on Stretch Marks

About 1 mg of the cosmetic composition prepared in Example 5 was topically applied to seven patients with stretch marks twice per day for a period of two months, and stretch marks were observed. The skin of the patients was photographed before, during and after the topical application of the cosmetic composition according to the present invention.

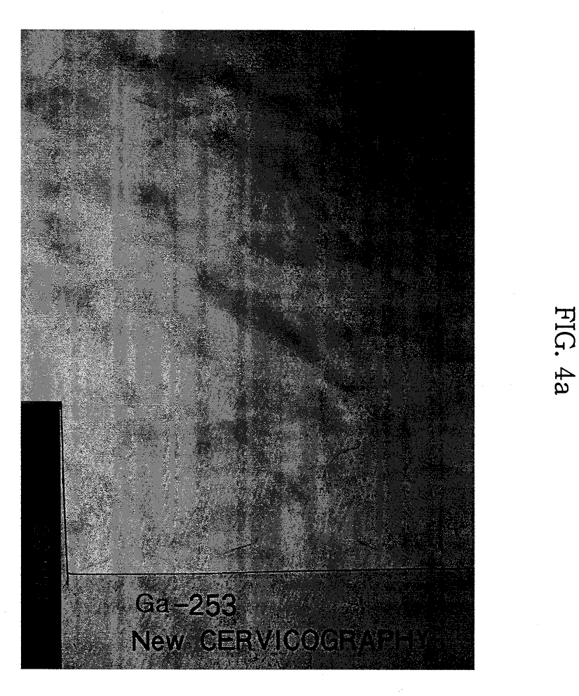

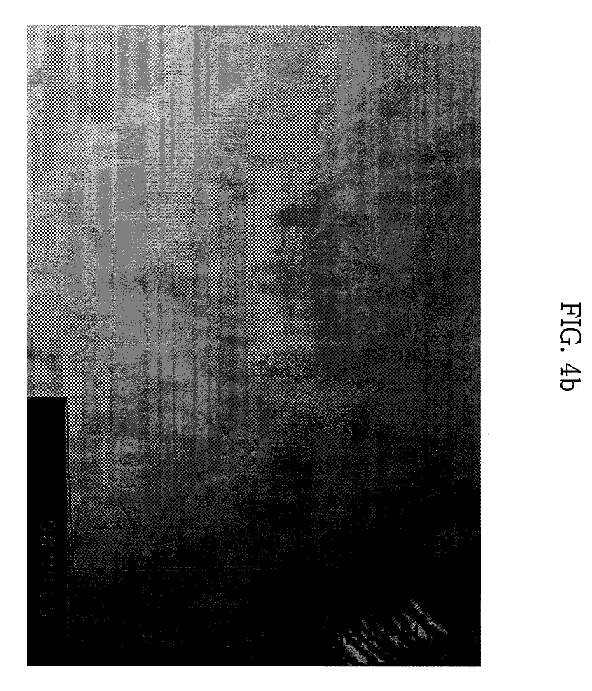

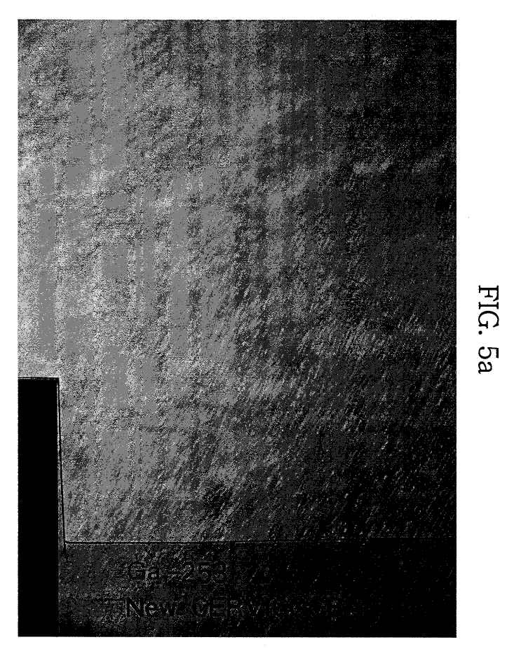

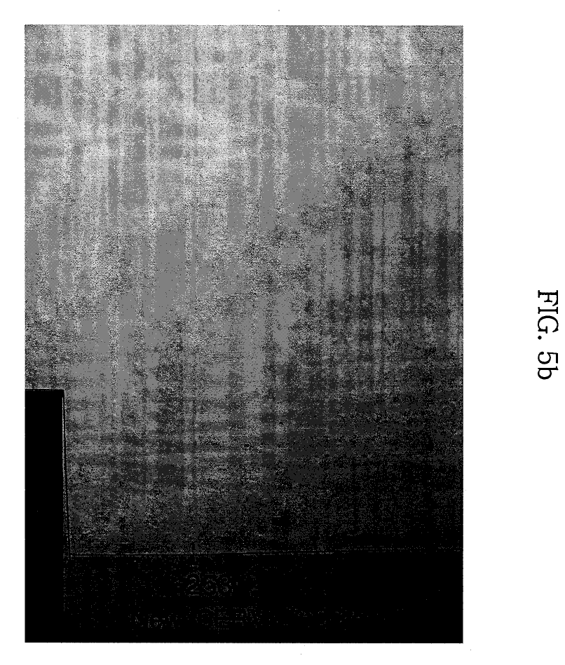

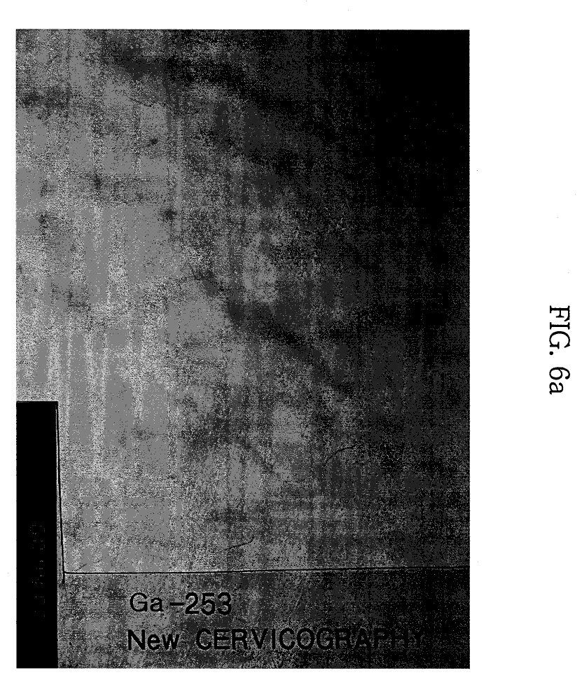

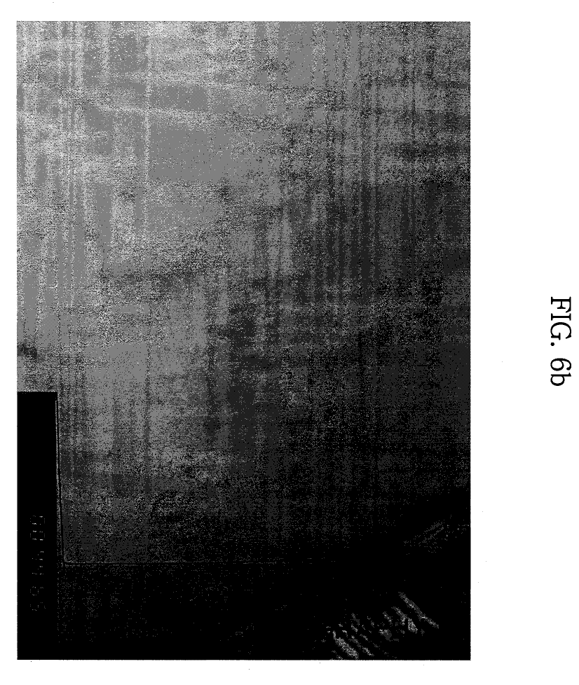

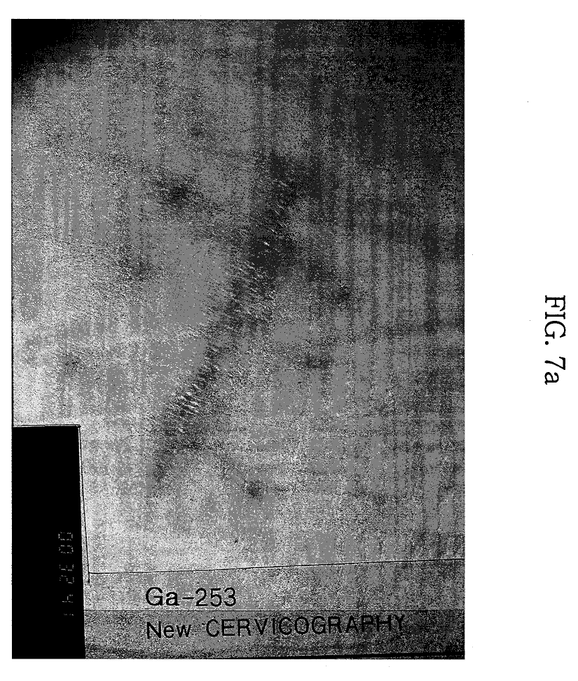

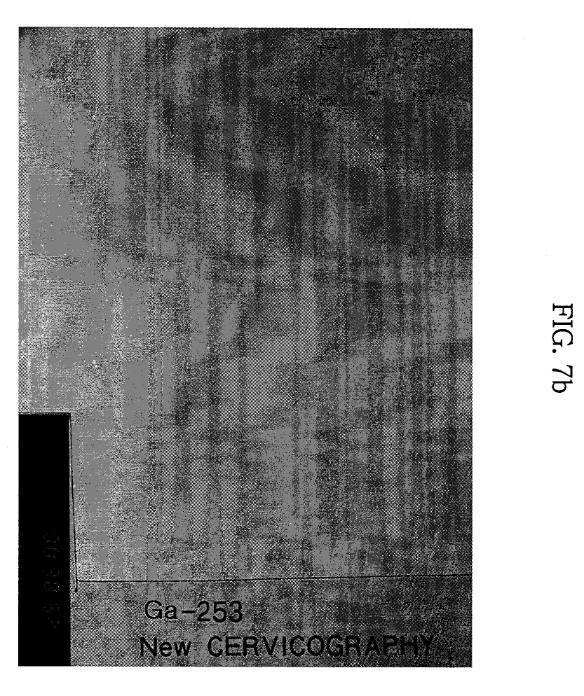

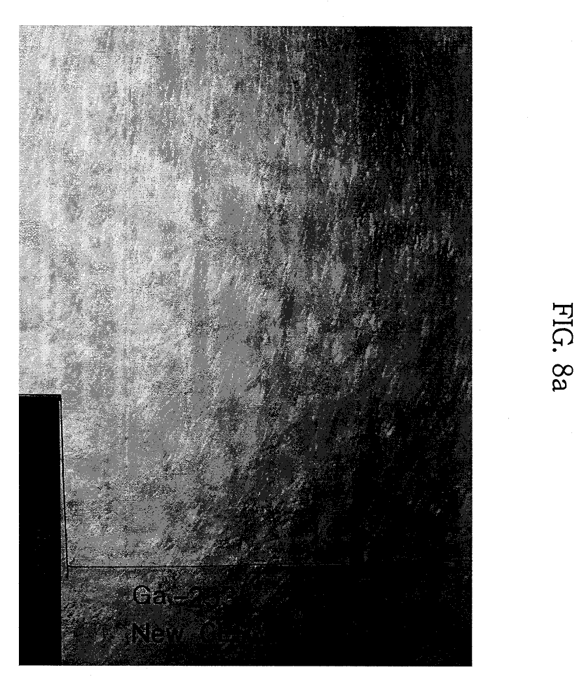

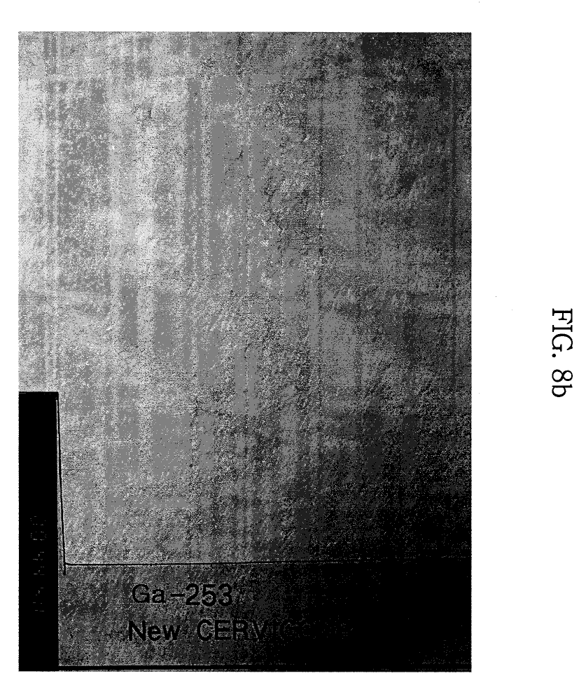

FIGS. 4a, 5a, 6a, 7a and 8a show the state of the skin where stretch marks occur before the topical application of the cosmetic composition according to the present invention. FIGS. 4b, 5b, 6b, 7b and 8b show the state of the skin where stretch marks occur after the topical application of the cosmetic composition according to the present invention. Stretch marks appearing as red or white bands were found to almost completely or completely disappear. These results indicate that the cosmetic composition according to the present invention reduces and even removes stretch marks.

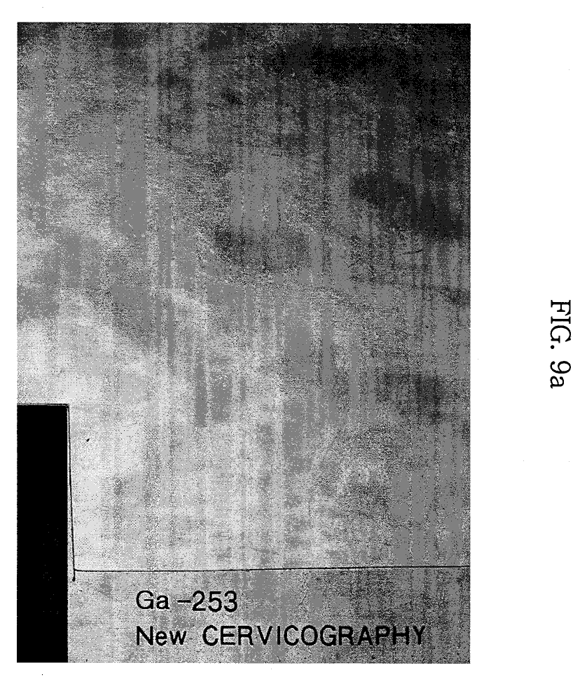

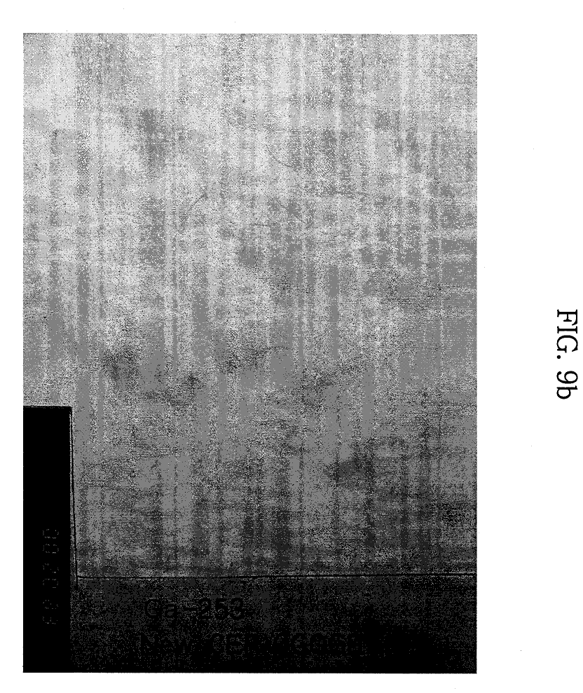

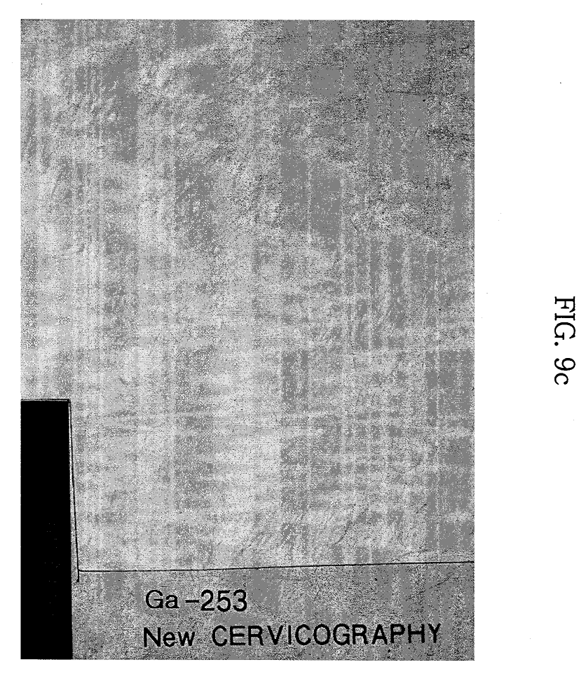

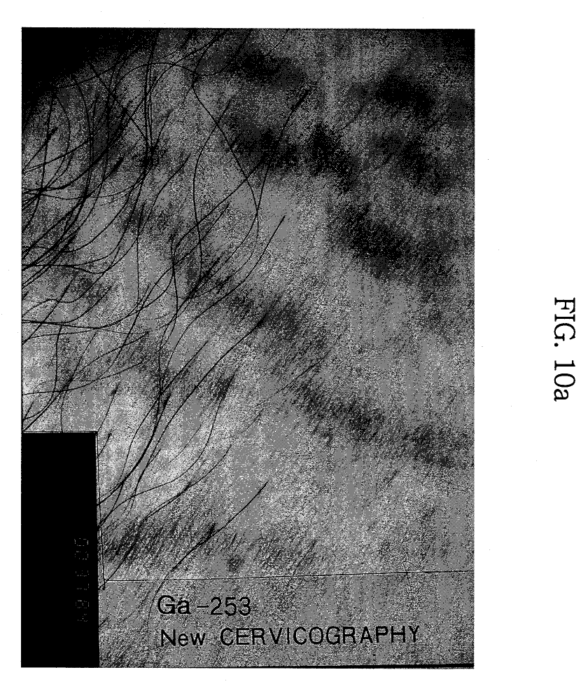

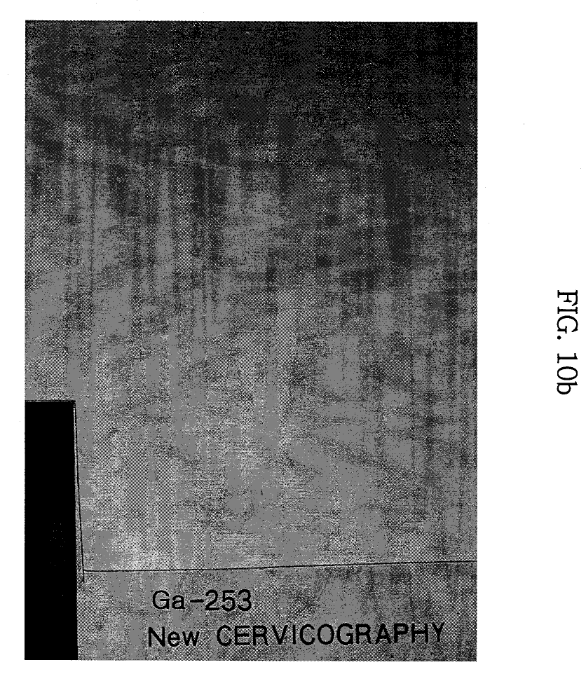

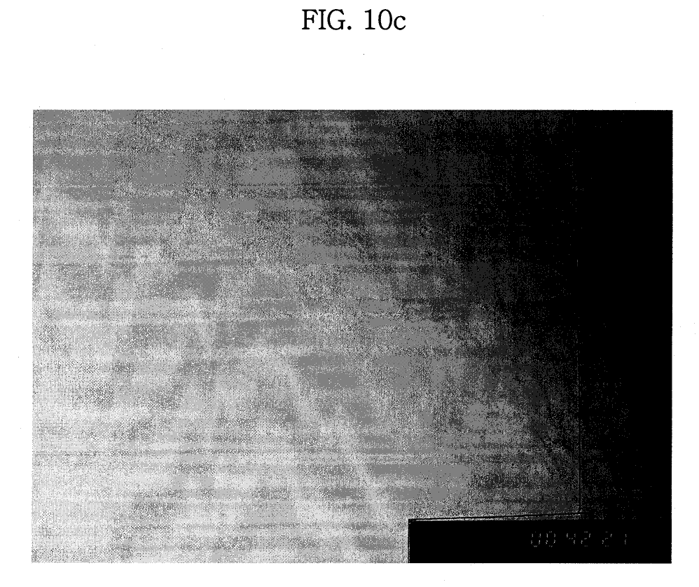

In addition, FIGS. 9a and 10a show the state of the skin where stretch marks occur before the cosmetic composition according to the present invention is applied to the skin. FIGS. 9b and 10b show the state of the skin after the cosmetic composition according to the present invention has been applied to the skin for about one month. FIGS. 9c and 10c show the state of the skin after the cosmetic composition according to the present invention has been applied to the skin for two months. Stretch marks were greatly reduced after the topical application of the cosmetic composition according to the present invention for about one month, and they were almost completely treated after the application period of two months.

### INDUSTRIAL APPLICABILITY

The composition according to the present invention is able to prevent and treat stretch marks by promoting regeneration of the skin through activation of keratinocytes and fibroblasts and stimulation of collagen synthesis.

1. A topically applied pharmaceutical composition for preventing and treating stretch marks, comprising citric acid, zinc and arginine as active components along with a pharmaceutically acceptable carrier.

2. The pharmaceutical composition for preventing and treating stretch marks according to claim 1, wherein the citric acid is present in an amount of 0.01 to 20% by weight based on the total weight of the composition, the zinc is present in an amount of 0.001 to 5% by weight based on the total weight of the composition, the arginine is present in an amount of 0.01 to 20% by weight based on the total weight of the composition, and the pharmaceutically acceptable carrier is present in a remaining amount to total 100% by weight.

3. A cosmetic composition comprising citric acid, zinc and arginine as active components along with a cosmetically acceptable carrier.

4. The cosmetic composition according to claim 3, wherein the citric acid is present in an amount of 0.01 to 20% by weight based on the total weight of the composition, the zinc is present in an amount of 0.001 to 5% by weight based on the total weight of the composition, the arginine is present in an amount of 0.01 to 20% by weight based on the total weight of the composition, and the cosmetically acceptable carrier is present in a remaining amount to total 100% by weight.
